(19) United States
(12) Patent Application Publication
(10) Pub. No.: US 2023/0312751 A1
(43) Pub. Date: Oct. 5, 2023

(54) Anti-Fibrotic Combination

(71) Applicant: Engitix Limited, London (GB)

(72) Inventors: Giuseppe Mazza, London (GB); Dennis Norman, London (GB); Massimo Pinzani, London (GB); Alan Holmes, London (GB); Gino Van Heeke, London (GB)

(21) Appl. No.: 18/021,734

(22) PCT Filed: Aug. 25, 2021
(86) PCT No.: PCT/EP2021/073514

§ 371 Date: Feb. 16, 2023

(30) Foreign Application Priority Data
Aug. 25, 2020 (GB) 2013270.0

(51) Int. Cl. C07K 16/40; A61P 1/16

(52) U.S. Cl. CPC C07K 16/40; A61P 1/16; C07K 2317/31; A61K 2039/505

(57) ABSTRACT

The invention relates to a method of treating or preventing a disease or condition associated with or characterised by fibrosis, comprising administering an effective amount of a chymase inhibitor simultaneously or sequentially with an effective amount of a tryptase inhibitor, to a subject in need thereof. Also provided are chymase and tryptase inhibitors for use in these methods.

### FIELD OF THE INVENTION

The present invention relates to a combination in the treatment and/or prevention of liver disease or conditions associated with fibrosis and particularly, although not exclusively, to the combination of a chymase inhibitor and a tryptase inhibitor in the treatment and/or prevention of liver diseases or conditions associated with fibrosis.

### BACKGROUND

Fibrosis, or the pathological deposition of connective tissue in response to damage or injury, resulting in the replacement of normal parenchymal tissue with connective tissue, is a symptom and cause of various conditions and diseases. Fibrosis can occur in various tissues of the body. In particular fibrosis of the liver is a major complication and key prognostic indicator for patients suffering from liver diseases.

Primary sclerosing cholangitis (PSC) is an idiopathic, heterogeneous, cholestatic liver disease that is characterised by persistent, progressive, biliary inflammation and fibrosis, and has a strong association with inflammatory bowel disease (IBD). The pathogenesis of PSC is poorly understood, although it is generally accepted that both genetic and environmental risk factors contribute to the development, progression and outcomes of the disease. Consequently, the identification of therapeutic targets and biomarkers has been difficult, and there is no effective medical therapy for this condition. End-stage liver disease necessitating liver transplantation may ultimately develop in affected patients. There remains a need for new, effective treatments for PSC.

Whilst the underlying causative mechanisms of PSC are unknown, mast cell (MC) numbers correlate positively with the severity of fibrosis (Jarido et al, 2017), and MC accumulation is reported at damaged bile ducts, around fibrous septa and portal tracts in PSC (Frances & Meininger, 2010). As mast cells are implicated in the inflammatory response, identifying mast cell factors in fibrotic tissue may provide putative drug targets for fibrotic conditions such as PSC.

### SUMMARY OF THE INVENTION

The invention relates to the discovery that mast cell-derived chymase and tryptase are present in far greater abundance in the extracellular matrix (ECM) of fibrotic tissue, and to the combination of a chymase inhibitor and a tryptase inhibitor in treating or preventing fibrosis.

Provided in a first aspect is a method of treating or preventing a disease or condition, comprising administering an effective amount of a chymase inhibitor and an effective amount of a tryptase inhibitor to a subject in need thereof. The chymase inhibitor is administered simultaneously or sequentially with the tryptase inhibitor.

Preferably, the disease or condition to be treated or prevented is a disease or condition associated with or characterised by fibrosis. In particular embodiments, the disease or condition is associated with or characterised by fibrosis of the liver with or without cholestasis. An exemplary condition is primary sclerosing cholangitis (PSC).

Also provided is a chymase inhibitor for use in the methods of treating or preventing a disease or condition according to the first aspect.

Further provided is a tryptase inhibitor for use in the methods of treating or preventing a disease or condition according to the first aspect.

Additionally, the foregoing provides a pharmaceutical composition comprising a chymase inhibitor and a tryptase inhibitor for use in the methods of treating or preventing a disease or condition according to the first aspect.

Further provided is the use of a chymase inhibitor and/or a tryptase inhibitor in the manufacture of a medicament for treating or preventing a disease or condition according to the first aspect.

Also provided is a kit comprising a chymase inhibitor and a tryptase inhibitor. This kit finds use in the methods of treating or preventing a disease or condition according to the first aspect.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1. Structure and composition of decellularised liver scaffolds, generated through the method outlined in WO2017017474A. (A) Macroscopic characterization of decellularization of human liver 3D scaffolds. Comparative microanatomy of fresh tissue and decellularised scaffolds demonstrates the structural integrity of the ECM in the decellularised scaffold. (AA) Macroscopic appearance of native cirrhotic liver 3D scaffold before and (AD) after decellularization. (AB, AC) Histological comparison of cirrhotic native tissue and decellularized 3D scaffold (AE, AF) after staining with Haematoxylin and Eosin (AE) showing acellularity (AE) and Sirius Red (SR) collagen preservation (AF), respectively (scale bars, 100-200 μm). (B) Microscopic characterization of decellularization of human liver 3D scaffolds. SEM imaging of native tissue (top panel, BQ-BS) and decellularized 3D cirrhotic scaffolds (bottom panel, BU-BW) showing the preservation of the nodular fibrotic ECM and conservation of the hepatocyte pockets (scale bars 50 μm, 10 μm, and 5 μm for each condition). (BT) Second harmonic generation analysis of fibrillar collagens structure (green) of healthy decellularized 3D scaffolds with (BX) more abundant and compact fibrillar collagen structures present in cirrhotic decellularized 3D scaffolds (scale bars, 20 μm). (C) Label-free quantitative proteomic analysis of proteins in the decellularised scaffolds is used to separate commonly expressed (87%) from those differentially expressed in healthy (7%) and cirrhotic (6%) liver in order to identify proteins characteristic of PSC liver as candidate targets.

FIG. 2. Comparative histological staining of healthy and PSC liver samples. A) Staining for mast cell marker cKit; B) staining for tryptase; C) staining for chymase; D) magnified view of chymase-stained PSC liver section demonstrating onion-like ring fibrosis.

FIG. 3. Chymase staining in the hepatic biliary tree. Localised increased presence of chymase can be seen in the epithelial cholangiocytes which define the intrahepatic biliary collecting ducts.

FIG. 4. Tryptase, chymase and mast-cell marker staining in healthy and disease-state liver tissues. A) Number of mast cell marker (c-Kit) positive cells in healthy (left, n-23), non-cirrhotic PSC (centre, n-25), and cirrhotic PSC (right, n=17). As can be seen, both PSC tissue types show higher numbers of c-Kit positive cells than healthy tissues, with more in cirrhotic than non-cirrhotic PSC. B) Number of Tryptase 20 positive cells in (from left to right) healthy (n=54), non-cirrhotic PSC (n-52), cirrhotic PSC (n=26), primary biliary cirrhosis (PBC) (n=10), and alcohol liver disease (ALD) (n=10). As can be seen, all disease state tissue types show higher numbers of tryptase-stained cells than healthy tissues. C) Number of CMA1 positive cells in (from left to right) healthy (n=49), non-cirrhotic PSC (n=48), cirrhotic PSC (n=24), PBC (n=10), and ALD (n=10). As can be seen, all disease state tissue types show higher numbers of chymase-stained cells than healthy tissues. Statistical significance is indicated by “***” (Kruskal-Wallis, p<0.001)

FIG. 5. Comparison of tryptase and chymase staining in disease-state liver tissues. There is a significant increase in tryptase- (p=0.0133, Kruskal-Wallis) and chymase-stained (p=0.0381, Kruskal-Wallis) cells in cirrhotic PSC compared to non-cirrhotic PSC. A) Number of tryptase positive cells in (from left to right) health (n=54), non-cirrhotic PSC (n=52), cirrhotic PSC (n=26), PBC (n=10) and ALD (n=10) samples as shown in FIG. 4B. B) Number of CMA1 positive cells in (from left to right) healthy (n=49), non-cirrhotic PSC (n=48), cirrhotic PSC (n=24), PBC (n=10), and ALD (n=10) samples as shown in FIG. 4C.

FIG. 6. Western blot of mast cell tryptase (MCT) in healthy (n=3), PSC (non-cirrhotic, n=10) and PSC (cirrhotic, n=10) liver tissue. A) Western blot for mast cell tryptase (31 KDa) and GAPDH (37 KDa) in healthy, PSC (non-cirrhotic) and PSC (cirrhotic) samples. B) Quantification of mast cell tryptase protein expression relative to GAPDH. C) Statistical comparison of mast cell tryptase fold protein expression relative to healthy tissue, where PSC (cirrhotic) tissue has a statistically significant increase in protein expression (p=0.0419, Kruskal-Wallis).

FIG. 7. Western blot of chymase (CMA1) expression in healthy (n=3), PSC (non-cirrhotic, n=10) and PSC (cirrhotic, n=10) liver tissue. A) Western blot for CMA1 (47KDa) and GAPDH (37 KDa) in healthy, PSC (non-cirrhotic) and PSC (cirrhotic) samples. B) Quantification of CMA1 protein expression relative to GAPDH. C) Statistical comparison of CMA1 fold protein expression relative to healthy tissue, where PSC (non-cirrhotic) and PSC (cirrhotic) tissue have a statistically significant increase in CMA1 protein expression relative to wild type (p=0.0312 and p=0.0206 respectively, Kruskal-Wallis).

FIG. 8. Western blot of collagen type I production by cells stimulated with chymase and/or tryptase. Primary human hepatic stellate cells (HSCs) were stimulated with exogenous addition of chymase (lanes 2-4) or tryptase (lanes (5-7) alone or in combination (lanes 8-10) and levels of deposited and cell associated type I collagen (upper panel) determined by western blot. Loading levels were confirmed by GAPDH (lower panel). Representative blot of n=3 primary human hepatic stellate cells assessed.

FIG. 9. Effects of chymase and tryptase on collagen deposition. Primary human hepatic stellate cells (HSCs) were stimulated exogenously with chymase (C) or tryptase (T) alone or in combination (C:T 100/1000), or TGFβ1 at 3 ng/mL, or maintained in serum-free conditions (SFM) and levels of deposited type I collagen determined by immunofluorescence. A. Microscopy imaging of treated cells. Top row (L to R): chymase treatment (100 ng/mL); tryptase treatment (1000, ng/mL); chymase plus tryptase treatment (C100/T1000 ng/mL), Bottom row: serum-free medium (SFM), TGF-beta1, no cell control. Chymase treatment results in altered collagen distribution patterns. B. Effect of treatments on cell number. Cell number in each well was determined by nuclear stain Hoechst. Chymase, tryptase or chymase and tryptase C100 ng/mL, T1000 ng/mL, C100/T1000 ng/mL, had no effect on cell number relative to the serum-free medium and TGF-beta control treatments. C Intensity of collagen type I immunofluorescence levels, normalised to cell number. Tryptase, or tryptase +chymase treatment, but not chymase when administered alone, results in increased collagen deposition.

FIG. 10. Effects of chymase and tryptase on mediators of fibrosis. Primary human HSCs were stimulated exogenously with chymase (C) at 300 ng/mL (C1); 100 ng/mL (C2) or 30 ng/mL (C3) or tryptase (T) at 3 mg/mL (T1); 1 mg/mL (T2) or 0.3 mg/mL (T3) alone or in combination C1+T1, C2+T2 or C3+T3, or TGFb1 at 3 ng/mL, or maintained in serum-free conditions (SFM) and levels of secreted IGFBP3 (A), Tenascin C (B), Collagen IV (C) or Endostatin (D) determined by Luminex.

FIG. 11. Effects of chymase and tryptase on TGFβ levels. Secreted Active (A) or latent (B) TGFβ levels by primary human HSCs stimulated exogenously with chymase (C) at 300 ng/mL (C1); 100 ng/mL (C2) or 30 ng/mL (C3) or tryptase (T) at 3 μg/mL (T1); 1 μg/mL (T2) or 0.3 μg/mL (T3) alone or in combination C1+T1, C2+T2 or 03+T3, or TGFβ1 at 3 or 1 ng/mL, or maintained in serum-free conditions (SFM) were determined in cells harbouring a TGFβ-responsive promoter linked to the firefly luciferase reporter gene.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

As used herein, “inhibitor” relates to a compound or substance which reduces or suppresses the activity or function of a target. A target may be one or more proteins or nucleic acids (such as mRNAs). An inhibitor may have multiple targets, which may share structural homology (e.g. in the case of related proteins) or a shared function (e.g. in the case of a pathway inhibitor). Inhibitors include a compound or substance which interacts with its target, for example a competitive or non-competitive/allosteric inhibitor. A competitive inhibitor competes with a substrate for the active site, whist an allosteric inhibitor binds to a site other than the active site and prevents substrate binding for example by stabilising a conformation which abolishes or disrupts the active form of the target. These interactions are typically reversible, however an inhibitor may be an irreversible inhibitor, for example an inhibitor which covalently links to a target, blocking or disrupting the active form. Alternatively, an inhibitor may be a transcriptional inhibitor, which reduces or abolishes the expression of a target, or an inhibitor may be a compound which promotes degradation of the target. An inhibitor may be a small molecule, a (poly) peptide or protein, a siRNA, a miRNA, or an antibody.

In some embodiments, the chymase inhibitor and tryptase inhibitor are comprised within the same entity, such that a single compound reduces or suppresses the activity or function of chymase and tryptase. For example, a chymase inhibitor as described above may be operably linked to a tryptase inhibitor as described above. Operably linked inhibitors need not be of the same type. For example, a small molecule inhibitor may be conjugated to another small molecule inhibitor, a small molecule inhibitor may be conjugated to an inhibitory antibody, or an inhibitory antibody may be conjugated to another inhibitory antibody. Operably linked inhibitors may be separated by a linker moiety. Alternatively, the method may utilise a bispecific chymase/tryptase inhibitor.

In some embodiments, the chymase inhibitor and tryptase inhibitor are provided as a bispecific antibody capable of binding and inhibiting both chymase and tryptase. A suitable bispecific antibody may be formed by conjugating together two antibodies or fragments thereof, optionally through a linker, through chemical cross-linking, or through recombinant means. Suitable bispecific antibody formats include (mab)₂, F(mab¹)₂, quadroma, bispecific diabody (bsDb), single-chain bispecific diabody (scBsDb), single-chain bispecific tandem variable domain (cBsTaFv), dock-and-lock trivalent Fab (DNL-(Fab)3) or a bispecific single-domain antibody (BssdAb).

As used herein, a “chymase inhibitor” is any compound or substance which reduces or suppresses the activity or function of chymase. A chymase inhibitor may also possess tryptase inhibitor activity.

Chymase is a serine protease that possesses chymotrypsin-like cleavage specificity. Humans possess a single α-chymase gene (CMA1), whereas rats and mice possess not only a single α-chymase gene, mouse mast cell protease-5 (mMCP-5) and rat mast cell protease-5 (rMCP-5), but also up to 14 β-chymase genes (Gallwitz and Hellman, 2006). Chymase has largely been documented for its involvement in angiotensin (Ang) II formation in cardiovascular disease (Doggrell and Wanstall, 2004; 2005). Chymase can also induce activation of TGF-β, a major regulator of tissue fibrosis, as well as MMP-9 (Takai et al., 2010), and has a putatively implicated role in inflammatory and functional gastrointestinal disorders (Heuston and Hyland, 2012). In some embodiments, chymase is α-chymase, preferably human α-chymase.

As used herein, a “tryptase inhibitor” is any compound or substance which reduces or suppresses the activity or function of tryptase. Tryptase is the most abundant secretory granule-derived serine proteinase contained in mast cells. In some embodiments, tryptase is a human tryptase selected from tryptase alpha-1, tryptase beta-1, tryptase beta-2, tryptase delta, tryptase gamma, and tryptase epsilon. Tryptase activity or function may be determined by ability to cleave a synthetic substrate N-benzoyl-D,L-arginine-p-nitroanilide (BAPNA), and a tryptase inhibitor may reduce BAPNA cleavage in an assay.

In some embodiments, a tryptase inhibitor reduces or suppresses the activity or function of one or more tryptases, preferably a human tryptase, selected from tryptase alpha-1, tryptase beta-1, tryptase beta-2, tryptase delta, tryptase delta, tryptase gamma and tryptase epsilon. Preferably, a tryptase inhibitor reduces or suppresses the activity or function of one or more tryptase, preferably a human tryptase, selected from tryptase alpha-1, tryptase beta-1 and/or tryptase beta-2.

In some embodiments, a tryptase inhibitor is a broad spectrum inhibitor which reduces or suppresses the activity or function of two or more, three or more, four or more, five or more, six or more, or all seven tryptases, preferably human tryptases, selected from tryptase alpha-1, tryptase beta-1, tryptase beta-2, tryptase delta, tryptase delta, tryptase gamma and tryptase epsilon. In some embodiments, broad spectrum inhibitor reduces or suppresses the activity or function of two or more, or all three tryptases, preferably human tryptases, selected from tryptase alpha-1, tryptase beta-1 and/or tryptase beta-2. Alternatively, a tryptase inhibitor may be specific for a single tryptase, preferably a human tryptase, selected from tryptase alpha-1, tryptase beta-1, tryptase beta-2, tryptase delta, tryptase delta, tryptase gamma, and tryptase epsilon, and shows substantially no inhibitory activity for the other tryptase in said list.

As used herein, “treatment” or “treating,” or “ameliorating” are used interchangeably. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For preventative or prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. “Preventing” also encompasses halting the progression of a disease, for example to an acute or chronic state.

“Patient”, “subject” or “subject in need thereof” refers to a living organism suffering from or prone to a disease or condition that can be treated by using the methods provided herein. The term does not necessarily indicate that the subject has been diagnosed with a particular disease, but typically refers to an individual under medical supervision. In preferred embodiments, a subject or patient is human.

As used herein, the term “administering” means oral administration, administration as a suppository, topical contact, sublingual, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g. to reduce metabolic degradation). The compositions of the present invention can be delivered transdermally, by a topical route, formulated as applicator sticks, solutions, suspensions, emulsions, gels, creams, ointments, pastes, jellies, paints, powders, and aerosols.

“Simultaneous” administration refers to administration of the agents together, for example as a pharmaceutical composition containing the agents (i.e. a combined preparation), or immediately after each other and optionally via the same route of administration, e.g. to the same artery, vein or other blood vessel. In particular embodiments, the chymase inhibitor and the tryptase inhibitor may be administered simultaneously in a combined preparation. In certain embodiments upon simultaneous administration the two or more of the agents may be administered via different routes of administration. Simultaneous administration may refer to administration at the same time, or within e.g. 1 hr, 2 hrs, 3 hrs, 4 hrs, 5 hrs, 6 hrs, 8 hrs, 12 hrs, 24 hrs, 36 hrs or 48 hrs.

“Sequential” administration refers to administration of one or more of the agents followed after a given time interval by separate administration of another of the agents. It is not required that the two agents are administered by the same route, although this is the case in some embodiments. The time interval may be any time interval, including minutes, hours, days, weeks, months, or years. Sequential administration may refer to administrations separated by a time interval of one of at least 10 min, 30 min, 1 hr, 6 hrs, 8 hrs, 12 hrs, 24 hrs, 36 hrs, 48 hrs, 3 days, 4 days, 5 days, 6 days, 1 week, 2 weeks, 3 weeks, 1 month, 6 weeks, 2 months, 3 months, 4 months, 5 months or 6 months.

Utilising the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

An “effective amount” is an amount sufficient to accomplish a stated purpose (e.g. achieve the effect for which it is administered, treat a disease, reduce enzyme activity, reduce one or more symptoms of a disease or condition, reduce viral replication in a cell). An example of an “effective amount” is an amount sufficient to contribute to the treatment, prevention, or reduction of a symptom or symptoms of a disease, which could also be referred to as a “therapeutically effective amount”. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose and may occur only after administration of a series of doses. Thus, a prophylactically effective amount may be administered in one or more administrations. An “activity-decreasing amount,” as used herein, refers to an amount of antagonist required to decrease the activity of an enzyme or protein relative to the absence of the antagonist. A “function-disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The compounds described herein may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, intratumoural, subcutaneous, intradermal, oral, or transdermal routes of administration which may include injection or infusion. Suitable formulations may comprise the antigen-binding molecule in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

The combination herein of a tryptase inhibitor and a chymase inhibitor finds use in the treatment or prevention of diseases of conditions characterised by fibrosis, preferably fibrosis of the liver.

As used herein, “fibrosis” refers to the excessive accumulation of extracellular matrix proteins including collagen, and development of scar tissue within an organ of the body. It occurs when repetitive or long-lasting injury or inflammation causes excessive amounts of scar tissue to build up in the organ. Fibrosis may be distinguished from inflammation. Inflammation is a biological response which occurs in many chronic diseases including autoimmune diseases such as rheumatoid arthritis and acute diseases such as sepsis and is characterised by the excessive recruitment of immune cells into tissues and/or immune cell activation and release of inflammatory mediators. In contrast, fibrosis is defined by the excessive deposition of extracellular matrix (ECM) proteins in tissues and organs which in turn leads to a loss of normal tissue architecture and ultimately a loss of organ function. Excessive ECM deposition in fibrosis is predominantly driven by the activation of tissue-resident mesenchymal cells. Whilst inflammation is a cardinal feature of all fibrotic diseases (and non-fibrotic chronic and acute diseases), it does not directly cause excessive ECM deposition rather it is understood to act in concert with other stimuli as a trigger to induce mesenchymal cells (such as hepatic stellate cells and fibroblasts) activation that in turn leads to excessive ECM deposition by these cells in the context of fibrotic diseases. Many distinct triggers can contribute to the development of progressive fibrotic disease. Examples include, inherited genetic disorders; persistent infections; recurrent exposure to toxins, irritants or smoke; chronic autoimmune inflammation; minor human leukocyte antigen mismatches in transplants; myocardial infarction; high serum cholesterol; obesity; and poorly controlled diabetes and hypertension Wynn T A Nat Med 2013 doi:10.1038. It is now clear that many components of the innate and adaptive immune system and its response participate in the differentiation and activation of these mesenchymal cells. Thus it follows that different treatments will be effective at reducing inflammation and fibrosis.

Regardless of the initiating events, a feature common to all fibrotic diseases is the activation of ECM producing mesenchymal cells, which are the key mediators of fibrotic tissue remodeling (Gabbiani G J Pathol 2003). For example, the left-ventricular hypertrophy that accompanies chronic hypertension is caused by an abnormal accumulation of ECM components such as collagen. This reactive and progressive interstitial fibrosis contributes to myocardial stiffness and, ultimately, ventricular dysfunction, and it is believed to result from the persistent activation of the resident mesenchymal cells, cardiac myofibroblasts.

In liver fibrosis caused by chronic hepatitis C virus (HCV) infection or alcohol abuse or nonalcoholic steatohepatitis (NASH) a similar activation of resident mesenchymal cells occurs eg hepatic stellate cells (Parola M Mol Aspects Med 2019). The excessive ECM deposition in the liver distorts normal liver tissue architecture, leading to hepatocellular dysfunction and increased hepatic resistance to blood flow, which cause hepatic insufficiency and portal hypertension.

In particular, fibrosis may be liver fibrosis. Liver fibrosis may be the result of a chronic disease or condition, including infections, damage, amyloidosis and cancer, affecting a tissue. A disease or condition associated with or characterised by fibrosis may be caused by fibrosis, or may result in fibrosis. In some embodiments, the disease or condition causes or can cause fibrosis as a side-effect.

In some embodiments, fibrosis is characterised or the stage of fibrosis predicted by increased expression or the presence of one or more markers associated with fibrosis in a tissue or organ affected by fibrosis relative to a reference for a healthy or unaffected tissue or organ. In some embodiments, fibrosis is characterised by increased levels or the presence of one or more markers associated with fibrosis in serum, blood, and/or urine relative to a reference value for a healthy or unaffected individual. A reference value may be directly determined in a sample from the patient (for example, from an earlier sample, or from a different organ or tissue), or from a healthy or unaffected patient. Alternatively, a reference value may be predetermined. The expression or presence of one or more markers may be determined in a sample, for example a biopsy sample, a blood sample, a serum sample, or a urine sample. The marker expression or presence may be intra- or extracellular. In some embodiments, fibrosis is characterised or the stage of fibrosis predicted by the increased presence of one or more markers associated with fibrosis in the ECM of a tissue or organ affected by fibrosis. Suitable markers include alpha smooth muscle actin (αSMA), collagen type 1 (COL1), tissue inhibitor of metalloproteinases 1 (TIMP-1), amino-terminal propeptide of type III procollagen (PIIINP), hyaluronic acid (HA), and Transforming growth factor beta (TGF-β).

In some embodiments, liver fibrosis is characterised or the stage of liver fibrosis predicted by the increased presence of marker set tissue inhibitor of metalloproteinases 1 (TIMP-1), amino-terminal propeptide of type III procollagen (PIIINP) and hyaluronic acid (HA) in the liver or in serum. This marker set may be referred to collectively as “Enhanced Liver Fibrosis” (ELF) score.

In some embodiments, fibrosis is characterised by elevated levels, preferably tissue or cell-associated levels, of chymase and/or tryptase relative to a reference for a healthy or unaffected individual.

Whilst fibrosis can result from prolonged and persistent inflammation, the two conditions may be distinguished thusly. In contrast to acute inflammatory reactions, which are characterised by rapidly resolving vascular changes, oedema and neutrophilic inflammation, fibrosis typically results from inflammation—defined as an immune response that persists for several months and in which inflammation, tissue remodeling and repair processes occur simultaneously. The fibrotic stage may therefore be characterised by the amount of scar tissue and its pattern of distribution, and substantial aberrant deposition of ECM material such as collagens, in which normal tissue is replaced with permanent scar tissue. Evaluation of the amount of inflammation is defined as “activity score” in some scoring systems like METAVIR for HCV and Brunt's for NASH and may be used to subcategorise or provide a prognosis of the development of the fibrosis. It is also possible to have fibrosis without inflammation, e.g. in tendinitis, such as lateral epicondylitis, where the fibrotic legion contains few inflammatory cells. Fibrosis, particularly liver fibrosis, may be distinguished from inflammation histologically.

The presence or extent of fibrosis in a tissue or organ can be assessed by histopathological methods, for example through the examination of a biopsy or surgical specimen obtained from the affected tissue. Preferred methods applicable to liver fibrosis include the METAVIR liver biopsy histological staging system, and morphometric methods (for example collagen proportionate area, CPA). In some embodiments, fibrosis can be additionally assessed through non-invasive methods (including serum marker levels, for example ELF levels, elastography, MRI) in order to characterise or allow a prediction of the stage of fibrosis.

In some embodiments, fibrosis may be further characterised or the stage of fibrosis predicted by mast cell accumulation in the tissue or organ affected by fibrosis. Mast cells are migrant cells of connective tissue containing granules rich in histamine and heparin, which induce inflammation and promote fibrosis and vascular cell activation. In some embodiments, mast cells accumulate in the tissue or organ affected by fibrosis to a level 2×, 5×, 10× or more than 10× that observed in the healthy or unaffected tissue or organ. In some embodiments, mast cell accumulation is characterised by increased mast cell degranulation levels relative to those observed in healthy or unaffected tissue or organ. Upon degranulation, tryptase is released from mast cells along with histamine, heparin, chymase, and other mast cell granule products. Degranulation may be characterised by increased levels of tryptase, histamine, heparin, and/or chymase in the tissue or organ relative to those observed in healthy or unaffected tissue or organ.

In some embodiments, fibrosis may be further characterised or the stage of fibrosis predicted by increased fibroblast proliferation in the tissue or organ affected by fibrosis relative to that observed in healthy or unaffected tissue or organ.

Fibrosis may be characterised or the stage of fibrosis predicted by increased accumulation of ECM in the tissue or organ affected by fibrosis relative to that observed in healthy or unaffected tissue or organ.

Where a comparison is made between the levels of an indicator of fibrosis (e.g. extent of fibrotic scarring, collagen deposition expression or presence of a marker associated with fibrosis, fibroblast proliferation) in a tissue or organ affected by fibrosis and a healthy or unaffected tissue or organ, the level observed in the tissue or organ affected by fibrosis may be compared to those directly measured in a healthy tissue or organ, or to a set of predetermined reference values for a healthy tissue or organ.

Exemplary diseases or conditions associated with or characterised by fibrosis include liver fibrosis (alcoholic, viral, autoimmune, metabolic and hereditary chronic disease), renal fibrosis (e.g., resulting from chronic inflammation, infections or type II diabetes), lung fibrosis (idiopathic or resulting from environmental insults including toxic particles, sarcoidosis, asbestosis, hypersensitivity pneumonitis, bacterial infections including tuberculosis, etc.), interstitial fibrosis, systemic scleroderma (autoimmune disease in which many organs become fibrotic), macular degeneration (fibrotic disease of the eye), pancreatic fibrosis (resulting from, for example, alcohol abuse and chronic inflammatory disease of the pancreas), fibrosis of the spleen (from sickle cell anemia, other blood disorders) cardiac fibrosis (resulting from infection, inflammation and hypertrophy), mediastinal fibrosis, myelofibrosis, endomyocardial fibrosis, retroperitoneal fibrosis, progressive massive fibrosis, nephrogenic systemic fibrosis, fibrotic complications of surgery, especially surgical implants, injection fibrosis and secondary conditions and disease states of fibrosis.

In some embodiments, the disease or condition to be treated or prevented is a disease or condition associated with or characterised by fibrosis of an organ selected from the liver, the heart, the kidney, the pancreas, the eye, the skin, the pancreas, the intestine, the brain, and/or the lung.

In some embodiments, the disease or condition to be treated or prevented is a disease or condition associated with or characterised by fibrosis of the heart (cardiac fibrosis). Fibrosis of the heart may be myocardial fibrosis, which is a common phenomenon associated the late stages of diverse cardiac diseases. The disease or condition associated with or characterised by fibrosis of the heart may be ischemic heart disease, an inherited cardiomyopathy mutation, myocardial ischemia, aortic stenosis, amyloidosis, Anderson-Fabry disease, endomyocardial fibrosis (EMF), cancer of the heart, or damage, a wound, or an injury to the heart.

In some embodiments, the disease or condition to be treated or prevented is a disease or condition associated with or characterised by fibrosis of the kidney (renal fibrosis). Diseases and conditions associated with or characterised by fibrosis of the kidney include chronic kidney disease, nephrotic syndrome, nephritic syndrome, tubulointerstitial renal fibrosis, retroperitoneal fibrosis, focal segmental glomerulosclerosis, inflammatory kidney disease, pyelonephritis, lupus nephritis, glomerulonephritis), cancer of the kidney, or damage, a wound, or an injury to the kidney.

In some embodiments, the disease or condition to be treated or prevented is a disease or condition associated with or characterised by fibrosis of the pancreas (pancreatic fibrosis). Diseases and conditions associated with or characterised by fibrosis of the pancreas include acute pancreatitis, chronic pancreatitis, exocrine pancreatic insufficiency (EPI), pancreatic cancer (for example, pancreatic ductal carcinoma), or damage, a wound, or an injury to the pancreas.

In some embodiments, the disease or condition to be treated or prevented is a disease or condition associated with or characterised by fibrosis of the lung (pulmonary fibrosis). Diseases and conditions associated with or characterised by fibrosis of the lung include idiopathic interstitial lung disease, idiopathic pulmonary fibrosis (IPF), desquamative interstitial pneumonia (DIP), acute interstitial pneumonia (AIP), nonspecific interstitial pneumonia (NSIP), respiratory bronchiolitis-associated interstitial lung disease (RB-ILD), cryptogenic organizing pneumonia, lymphoid interstitial pneumonia, pulmonary fibrosis resulting from an autoimmune disease, pulmonary fibrosis resulting from an inhaled substance (for example, asbestosis or silicosis), pulmonary fibrosis resulting from a pulmonary infection (for example, pneumonia, tuberculosis, bronchiolitis), lung cancer (for example, small-cell lung carcinoma or non-small-cell lung carcinoma), or damage, a wound, or an injury to the lung.

In some embodiments, the disease or condition to be treated or prevented is a disease or condition associated with or characterised by fibrosis of the eye. Such a disease may be selected from glaucoma, pterygia, retinal fibrosis, fibrovascular scarring of the eye, subretinal fibrosis, age-related macular degeneration (ARMD), epiretinal fibrosis, and diabetic retinopathy. In some embodiments, fibrosis of the eye is capsular fibrosis after cataract surgery.

In some embodiments, the disease or condition to be treated or prevented is a disease or condition associated with or characterised by fibrosis of the skin. The disease or condition may be selected from chronic dermal fibrosis, scleroderma (e.g. localised scleroderma, preferably scleroderma-systemic sclerosis), telangiectasia, dermatofibrosis, eosinophilic fasciitis, the eosinophilia-myalgia syndrome, scleromyxedema (papular mucinosis). In some embodiments, the condition to be treated or prevented is a skin wound or scar.

In some embodiments, the disease or condition to be treated or prevented is a disease or condition associated with or characterised by intestinal fibrosis. In some embodiments, the intestinal fibrosis is associated with inflammatory bowel disease (IBD). In some embodiments, IBD is selected from ulcerative colitis and Crohn's disease.

In some embodiments, the disease or condition to be treated or prevented is associated with or characterised by fibrosis of the liver (hepatic fibrosis). Diseases that may be associated with or characterised by fibrosis of the liver include alcoholic liver disease (ALD), primary sclerosing cholangitis (PSC), primary biliary cirrhosis (PBC), drug-induced hepatocellular fibrosis, viral hepatitis, autoimmune hepatitis, metabolic disease associated hepatocellular fibrosis, diabetes mellitus, hereditary chronic disease associated hepatocellular fibrosis, liver cancer, or damage, a wound, or an injury to the liver.

The stage or extent of fibrosis in the liver may be defined using the Metavir scale (Bedossa et al, 1996), which stratifies fibrosis based on liver biopsy to stages 0 to 4 as follows: F0—no fibrosis, F1—portal fibrosis, F2—periportal fibrosis, F3—bridging fibrosis, F4—cirrhosis. In some embodiments, the liver disease or condition is associated with or characterised by fibrosis of the liver at stage F1 or higher, stage F2 or higher, stage F3 or higher, or stage F4, according to the Metavir scale. In some embodiments, fibrosis of the liver has a staging of F1 to F3, F2 to F3, or F3, in which case the fibrosis may be called “pre-cirrhotic”.

In some embodiments, the fibrosis of the liver is associated with elevated serum levels of hyaluronic acid (HA), amino-terminal pro-peptide of type III pro-collagen (PIIINP), and/or tissue inhibitor of metalloproteinase 1 (TIMP1). This characterisation may be performed using the Enhanced Liver Fibrosis (ELF) test described Rosenberg W et al., Gastroenterology 2004

In some embodiments, the disease or condition is associated or characterised by cirrhosis of the liver. Cirrhosis is the result of acute fibrosis and scarring in the liver, and significantly impacts liver function. Cirrhosis is defined as F4 on the Metavir scale. In some embodiments, the disease or condition to be treated or prevented is cirrhosis of the liver.

In some embodiments, the disease or condition to be treated or prevented is a liver disease or condition associated with or characterised by cholestasis. Cholestasis is the result of a decrease in bile flow due to obstruction of bile flow through intra-or extrahepatic bile ducts, resulting in substances normally excreted into bile being retained within the liver. It may be the result of persistent injury of cholangiocytes (the cells lining the bile ducts) which results in constriction, inflammation and fibrosis of the bile duct and biliary tree. Cholestasis may be associated with hepatocellular necrosis/apoptosis, hepatocellular membrane lipid peroxidation, a pro-fibrogenic effect on hepatic stellate cells, chronic wound healing, and/or fibrosis. Cholestasis frequently results in cholangiocarcinoma, and the methods and combination as described herein may be used for treating or preventing cholangiocarcinoma, e.g. in a patient suffering from cholestasis.

In some embodiments, the disease or condition to be treated or prevented is primary sclerosing cholangitis (PSC). PSC is characterised by persistent, progressive biliary inflammation, resulting in cholestasis and hepatocellular fibrosis. Whilst the pathology of PSC is poorly understood, several subtypes of primary sclerosing cholangitis have been characterised. In some embodiments, the PSC to be treated or prevented is the “classic PSC” subtype i.e. PSC which involves the entire biliary tree. In other embodiments, the PSC to be treated or prevented is “small duct PSC” i.e. affects only small intrahepatic bile ducts. Further subtypes may be identified by association with other conditions. In some embodiments, PSC is associated with autoimmune hepatitis. In some embodiments, PSC is associated with inflammatory bowel disease. In other embodiments, PSC is not associated with inflammatory bowel disease. Subtypes of PSC ay also be characterised by their association with one or more markers. In some embodiments, PSC is associated with increased serum IgG4 levels. Approximately 10% of patients with primary sclerosing cholangitis have increased serum IgG4 levels, and these patients have a poorer outcome than those with normal serum IgG4 levels (Mendes et al, 2006).

PSC is described in detail in Karlsen et al 2017, Journal of Hepatology vol. 67 pp. 1298-1323, and Lazaridis and La Russo, NEJM 2016.

In some embodiments, PSC is associated with fibrosis of the liver as defined herein and additionally one or more of the following: abnormal serum alkaline phosphatase (ALP) levels, preferably elevated ALP levels, pancreatic autoantibody positivity, low serum albumin, elevated serum bilirubin, preferably elevated bilirubin over at least 3 months, elevated IgG4 and/or IgG3/IgG1 ratio, elevated serum IL8, elevated levels of vascular adhesion protein-1, dominant stenosis (preferably characterised by a bile duct diameter 1.5 mm smaller than that of the common duct or 1.0 mm smaller than that of a hepatic duct within 2 cm of the bifurcation at ERC), pruritus, jaundice, cholangitis, cholangiocarcinoma, and/or the presence in the serum of one or more autoantibody selected from anti-nuclear antibodies, anti-liver kidney microsomal antibodies, antibodies against soluble liver antigen/liver pancreas (anti-SLA/LP), anti-smooth muscle antibodies, and/or elevated immunoglobulin G.

PSC may be categorised by association with one or more prognostic indices. Preferred prognostic indices are non-invasive, preferably serum levels of one or more biomarkers. In some embodiments, PSC is associated with abnormal serum alkaline phosphatase (ALP) levels, preferably elevated ALP levels. In some embodiments, PSC is associated with low serum albumin. In some embodiments, PSC is associated with elevated serum bilirubin, preferably elevated bilirubin over at least 3 months. In some embodiments, PSC is associated with elevated IgG4 and/or IgG3/IgG1 ratio. In some embodiments, PSC is associated with elevated serum IL8. In some embodiments, PSC is associated with elevated levels of vascular adhesion protein-1.

In some embodiments, PSC is associated with dominant stenosis. Dominant stenosis may be characterised by a bile duct diameter 1.5 mm smaller than that of the common duct or 1.0 mm smaller than that of a hepatic duct (within 2 cm of the bifurcation at ERC).

In some embodiments, PSC is associated with pruritus, jaundice, and/or cholangitis. In some embodiments, PSC has features of autoimmune hepatitis, and is associated with the presence in the serum of one or more of anti-nuclear antibodies, anti-liver kidney microsomal antibodies, antibodies against soluble liver antigen/liver pancreas (anti-SLA/LP), anti-smooth muscle antibodies, and/or elevated immunoglobulin G. In preferred embodiments, PSC is associated with, or is indicative of, cholangiocarcinoma. PSC is the main predisposing cause of cholangiocarcinoma, with an incidence 400-fold higher than the normal population. As such, any treatment for PSC may be viewed as a prophylactic treatment for cholangiocarcinoma.

In some embodiments, the disease or condition to be treated or prevented is secondary sclerosing cholangitis (SSC). SSC is similar to PSC, but has a causal agent outside the liver. In some embodiments, SSC is associated with or caused by a condition selected from: cholangiocarcinoma, IgG4-associated sclerosing cholangitis, HIV infection, sarcoidosis, choledocholithiasis, traumatic or ischaemic biliary injury, ampullary or pancreatic cancer, chronic pancreatitis, hilar lymphadenopathy, congenital choledochal cysts or biliary atresia, Chronic biliary infestation (e.g. liver fluke or ascaris), recurrent pyogenic cholangitis, choledochal varices (e.g. portal biliopathy), and/or ischaemic cholangiopathy

In some embodiments, the disease or condition to be treated or prevented is primary biliary cirrhosis (PBC). PBC is an autoimmune disease of the liver in which the bile ducts in the liver become damaged and may be subcategorised into four stages: Stage 1—characterised by inflammation and damage to the walls of medium-sized bile ducts; Stage 2—characterised by blockage of the small bile ducts; Stage 3—this stage marks the beginning of scarring; Stage 4—cirrhosis has developed, there is permanent, severe scarring and damage to the liver. In some embodiments, the PBC to be treated is stage 1. In some embodiments, the PBC to be treated is stage 2. In some embodiments, the PBC to be treated is stage 3. In some embodiments, the PBC to be treated is stage 4.

In some embodiments, the disease or condition to be treated or prevented is secondary biliary cirrhosis (SBC). Secondary biliary cirrhosis differs from primary biliary cirrhosis in that the bile ducts are obstructed or damaged due to another cause, such as a tumour, resulting in fibrosis and damage to the liver. The therapeutic approach disclosed herein may be effective to treat or prevent the fibrosis, and may be combined with a second therapy to treat or prevent the cause of the bile duct obstruction or damage.

In some embodiments, the disease or condition to be treated or prevented is alcoholic liver disease (ALD). ALD may be subcategorised into three main stages—alcoholic fatty liver disease, alcoholic hepatitis, and ALD characterised by cirrhosis. In some embodiments, the ALD to be treated is associated with alcoholic hepatitis and/or ALD characterised by cirrhosis.

In some embodiments, the disease or condition to be treated or prevented is non-alcoholic steatohepatitis (NASH). NASH is associated with a build-up of fat in the liver cells, resulting in inflammation and fibrosis. Prolonged, untreated NASH may result in cirrhosis. In some embodiments, NASH is associated with cirrhosis. In some embodiments, however, NASH is pre-cirrhotic.

In some embodiments, the disease or condition to be treated or prevented is selected from: liver fibrosis, lung fibrosis, kidney fibrosis, cardiac fibrosis, inflammatory bowel disease, and/or scleroderma.

In some embodiments, the disease or condition to be treated or prevented is biliary atresia. Biliary atresia affects infants, where the bile ducts become inflamed and blocked soon after birth, which if untreated results in cholestasis, fibrosis, and eventually cirrhosis.

Also provided herein is a method of determining whether a patient will respond to a treatment comprising an effective amount of a chymase inhibitor administered simultaneously or sequentially with an effective amount of a tryptase inhibitor, comprising diagnosing the presence of fibrosis, or of a disease or condition associated with fibrosis, in a tissue as outlined above, and selecting the patient as responsive to the treatment if the diagnosis is positive.

Also provided herein is a method of treating or preventing fibrosis, or of a disease or condition associated with fibrosis, in a patient comprising diagnosing the presence of fibrosis, or of a disease or condition associated with fibrosis, in a tissue obtained from the patient, and administering a treatment comprising an effective amount of a chymase inhibitor administered simultaneously or sequentially with an effective amount of a tryptase inhibitor according to the method outlined above.

Also provided herein is a kit comprising a chymase inhibitor and a tryptase inhibitor. Said kit finds use for treating or preventing a disease or condition associated with or characterised by fibrosis in a subject. The kit may further comprise instructions for administering an effective amount of the chymase inhibitor simultaneously or sequentially with an effective amount of the tryptase inhibitor to the subject.

Methods according to the present invention may be performed, or products may be present, in vitro, ex vivo, or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms. “Ex vivo” refers to something present or taking place outside an organism, e.g. outside the human or animal body, which may be on tissue (e.g. whole organs) or cells taken from the organism.

Pharmaceutical compositions may be prepared using a pharmaceutically acceptable “carrier” composed of materials that are considered safe and effective. “Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In some embodiments, this term refers to molecular entities and compositions approved by a regulatory agency of the US federal or a state government, as the GRAS list under section 204(s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans.

The term “carrier” refers to diluents, binders, lubricants and disintegrants. Those with skill in the art are familiar with such pharmaceutical carriers and methods of compounding pharmaceutical compositions using such carriers.

The pharmaceutical compositions provided herein may include one or more excipients, e.g., solvents, solubility enhancers, suspending agents, buffering agents, isotonicity agents, antioxidants or antimicrobial preservatives. When used, the excipients of the compositions will not adversely affect the stability, bioavailability, safety, and/or efficacy of the active ingredients, i.e. the chymase inhibitor and the tryptase inhibitor used in the composition. Thus, the skilled person will appreciate that compositions are provided wherein there is no incompatibility between any of the components of the dosage form. Excipients may be selected from the group consisting of buffering agents, solubilizing agents, tonicity agents, chelating agents, antioxidants, antimicrobial agents, and preservatives.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1—Chymase and Tryptase are Present in PSC Decellularised Scaffold

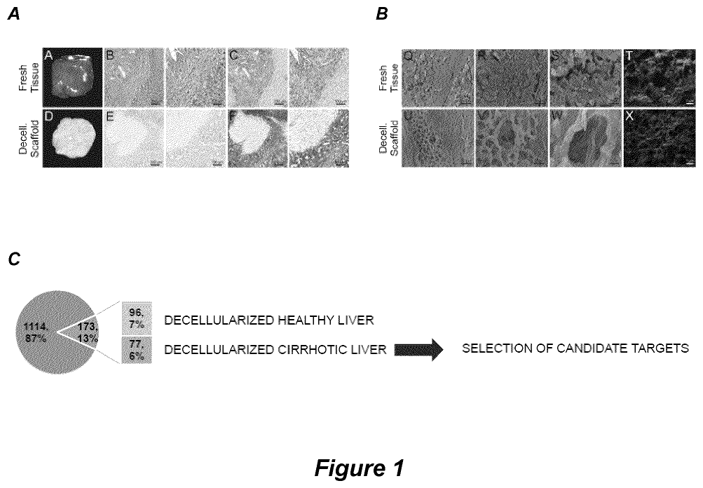

Decellularised liver scaffolds were generated for healthy and cirrhotic PSC human liver samples as outlined in WO2017017474A. Comparative microanatomy of fresh tissue and decellularised scaffolds is shown FIG. 1A, demonstrating the structural integrity of the ECM in the decellularised scaffold.

Label-free quantitative proteomic analysis was used to identify unique proteins in the decellularised scaffolds, in order to identify proteins characteristic of healthy and PSC liver.

A total of 1287 proteins were detected in either healthy or cirrhotic PSC decellularised scaffold. Of these, 173 (13%) showed a significant difference in enrichment between healthy and PSC decellularised scaffold (p<0.05, Student's t-test). Of these, 77 (6%) proteins were significantly enriched in PSC decellularised scaffold relative to healthy decellularised scaffold, whilst 96 (7%) of proteins were enriched in healthy decellularised scaffold relative to PSC decellularised scaffold. Proteins significantly enriched in PSC decellularised scaffold relative to healthy decellularised scaffold were selected as candidate therapeutic targets.

Notably, mast cell tryptase and chymase were found to be strongly upregulated in cirrhotic PSC ECM compared to healthy ECM (p-values: 0.0004 and 0.0007, respectively).

Thus, for the first time, chymase and tryptase were shown to be simultaneously present in decellularised scaffolds from fibrotic liver tissue.

### Example 2—Sample Providence and Qualitative Immunohistochemistry

Liver tissue samples from heathy and disease-state cohorts were acquired as outlined in Table 1.

TABLE 1
Sample cohorts (healthy vs non-healthy)

| | | PSC | | | |
|---|---|---|---|---|---|
| | | (non- | PSC | PBC | ALD |
| | Healthy | cirrhotic) | (cirrhotic) | (cirrhotic) | (cirrhotic) |
| Oslo | — | 27 | 17 | — | — |
| University | | | | | |
| Royal | 30 | 26 | 11 | 10 | 10 |
| Free | | | | | |
| Hospital | | | | | |
| Engitix | 24 | — | — | — | — |
| Total | 54 | 53 | 28 | 10 | 10 |

Formalin-fixed paraffin embedded tissues were retrieved from archived biopsy material in the Royal Free hospital as part of a research collaboration with the Hepatopathology research team at the Royal Free London NHS Foundation trust.

Samples of healthy and PSC livers were fixed in 10% neutral buffered formalin (NBF) for between 24 and 72 hours before paraffin processing. The tissue was processed via a standard 11-12 h paraffin processing schedule. In brief, the tissue was taken through 6 graded industrial denatured alcohols (IDA) 70-100% (aq) for 5 hours, 3 mixed polymer xylenes for 3 hours and 3 changes of paraffin wax (3 hours). The tissues were embedded in paraffin wax.

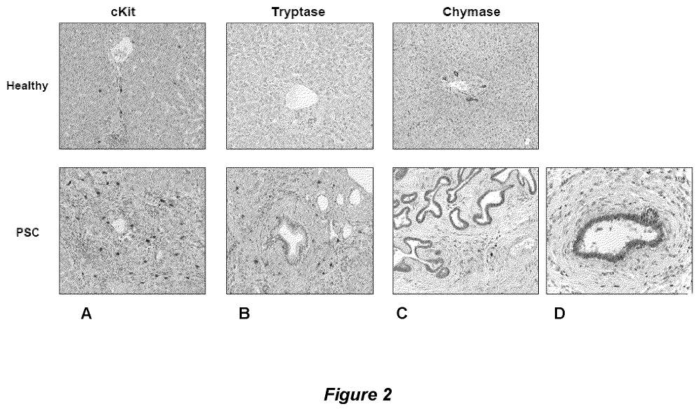

The presence of mast cells and the mast cell proteases tryptase and chymase in PSC liver was confirmed through immunohistochemical staining (see FIG. 2) as follows:

Sections were cut at 4 microns on Leica X-tra adhesive slides from the formalin-fixed paraffin embedded tissue. Slides were incubated overnight ˜16 hours at 37-40 C. All stainings with all antibodies were preformed simultaneously to avoid batch-to-batch staining variations.

Slides were dewaxed in 3 changes of Xylene over the course of 10 minutes. Slides were taken to water through 3 changes of industrial denatured alcohol (IDA) over the course of 5 mins, before being placed in water and checked by eye to ensure adequate dewax+hydration had occurred.

Antigen retrieval was performed based on antibody optimisation as follows:
c-Kit: slides were placed in 1 L pH 9.0 Tris-EDTA buffer at room temperature with a loosely fitted lid to allow gas to escape. It was then heated at 640W in a microwave for 20 mins, then the lid removed and allowed to cool at room temperature for 10 mins. The slides were then transferred quickly to tap water.
MCT: no antigen retrieval step was required.
CMA: slides were placed in 1 L pH 6.0 sodium citrate buffer at room temperature with a loosely fitted lid to allow gas to escape. It was then heated at 640 W in a microwave for 15 mins, then the lid removed and allowed to cool at room temperature for 10 mins. The slides were then transferred quickly to tap water.

Next, slides were placed in a humidity chamber with pH 7.6 TBS-Tween buffer and then blocked for endogenous H₂0₂ using the peroxide block from the Novolink Max Polymer detection kit (RE7280-K) for 5 mins, then washed in TBS for 5 mins. Non-specific binding of the post-primary was blocked using the Protein block from the Novolink Max Polymer detection kit.

Primary antibodies were diluted in the following concentrations in Leica IHC diluent (RE7133) and added to the slides for 60 mins, following which they were washed for 5 mins in pH 7.6 TBS-Tween:
c-Kit—1:300 (Dako A4502)
MCT—Neat (Leica PA0019)
CMA—1:200 (MyBioSource (MBS176889)

The slides were incubated in the post primary from the Novolink Max Polymer detection kit for 30 mins then washed in pH 7.6 TBS-Tween for 5 mins. The slides were then developed using the DAB from the Novolink Max Polymer detection kit (20 μL chromogen: 1000μL substrate buffer) then washed in pH 7.6 TBS-Tween for 5 mins. The slides were counter-stained in Mayer's Haematoxylin for 1-3 mins.

Slides were dehydrated through IDA and cleared in xylene over the course of 10 mins before mounting in DPX.

Images were taken on a Zeiss AxioSkop 50 microscope using Zeiss Achronplan objectives and a Zeiss ICc5 Axiocam camera.

In addition to more visible cKit, tryptase and chymase staining in PSC livers, fibrotic septa can be seen in tissues (dark spots) as well as onion-like ring fibrosis in PSC.

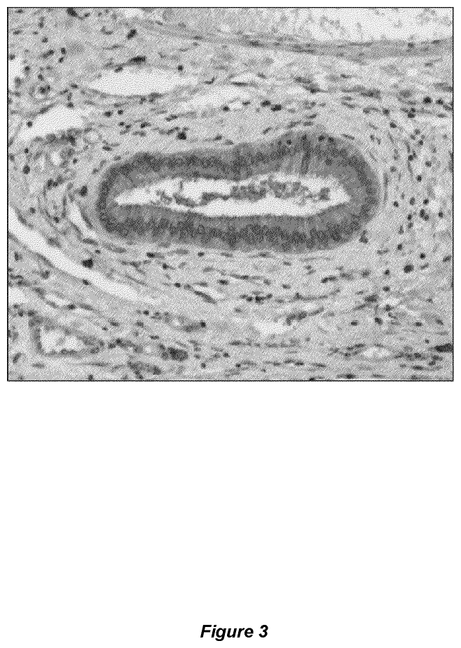

Chymase staining also revealed the accumulation of chymase in the intrahepatic biliary collecting ducts (FIG. 3).

This demonstrates that the PSC samples are fibrotic, and that they have increased mast cell, tryptase and chymase levels relative to healthy tissue.

### Example 3—Quantitative Immunohistochemistry

The presence of tryptase, chymase and mast-cell marker cKit was determined in liver tissue samples from healthy, PSC, PBC and ALD tissues.

Tissues were obtained and slides prepared as in Example 2. Slides were scored for positively stained cells in the portal and septal areas as follows:
1. Each slide was assessed for the most concentrated populations of positive cells i.e. hotspots, subjectively but blindly assessed. Then the field with the maximum number of positive cells and the associated portal tracts or fibrous septa (in cirrhotic livers) were imaged at ×20 objective magnification.
2. Three fields were considered the minimum for consideration with 5 fields desirable (86% of cases had 5 fields).
3. Positively stained cells were counted in 1 contiguous area of stroma.
4. Positively stained non-biliary non-hepatocyte cells in the stroma were counted.
5. Data was recorded on the number of positive cells per field, and area of the stroma in which the cells were found. From this the density of MCT/CMA1/c-Kit positive cells could be calculated as positive cells/mm².

Bile duct and parenchyma (hepatocyte) staining was graded as none/weak/moderate/strong intensity.

### Results:

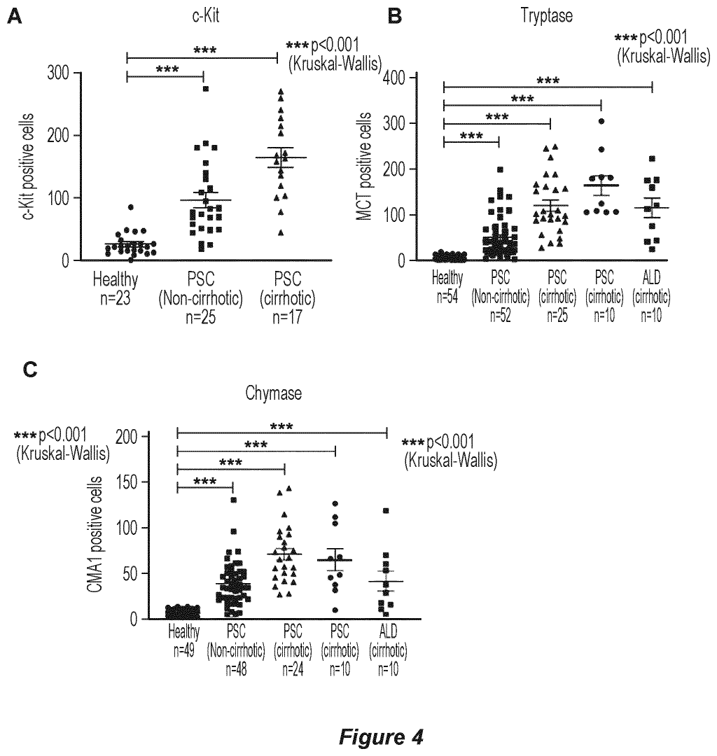

As shown in FIG. 4A, there was a significant increase of mast cells in non-healthy tissues compared to healthy tissues.

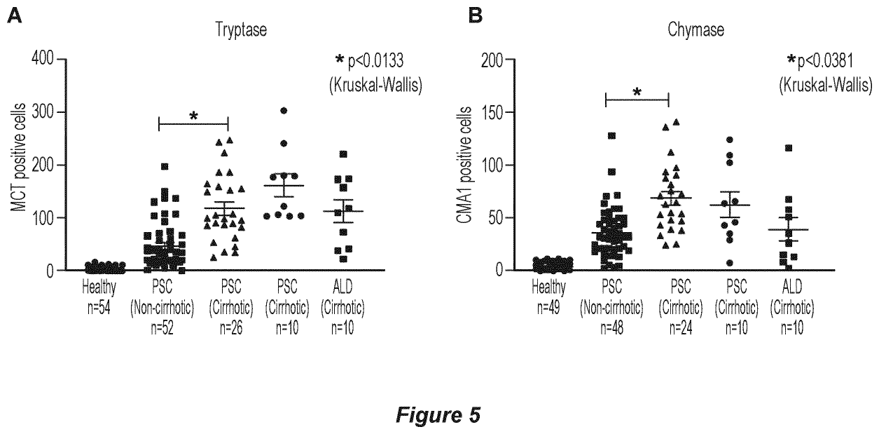

As can be seen in FIGS. 4B and 4C, there was a significant increase in tryptase-stained cells and chymase-stained cells in non-healthy tissues (PSC, PBC, and ALD) compared to healthy tissues, demonstrating that tryptase and chymase are indicative of disease states. Moreover, as seen in FIG. 5, there was a significant increase in tryptase and chymase-stained cells in cirrhotic PSC compared to non-cirrhotic PSC. This shows that tryptase and chymase levels can be used to determine disease progression.

### Example 4—Determination of MCT and CMA1 Expression in Liver Tissues by Western Blot

The expression of mast cell tryptase and CMA1 was quantified in healthy, non-cirrhotic PSC, and cirrhotic PSC whole livers through western blot.

Sample subgroups were obtained as follows: healthy liver tissues with <5% fat (n=3), PSC (Non-cirrhotic, n=10), and PSC (Cirrhotic, n=5).

Protein was extracted and quantified by micro-BCA assay to achieve an equal sample loading.

Expression levels of MCT, CMA1 and GAPDH in the samples were probed and measured by western blot, and protein quantified by densitometry. MCT and CMA1 levels were normalised to glyceraldehyde 3-phosphate dehydrogenase (GAPDH) expression. Three healthy, 10 non-cirrhotic PSC and 5 cirrhotic PSC samples were selected for analysis from those outlined above in Table 1, and expression levels of all three of MCT, CMA1, and GAPDH were determined in each of these samples.

Data were not all normally distributed; statistical analysis was performed using Kruskal-Wallis (non-parametric test).

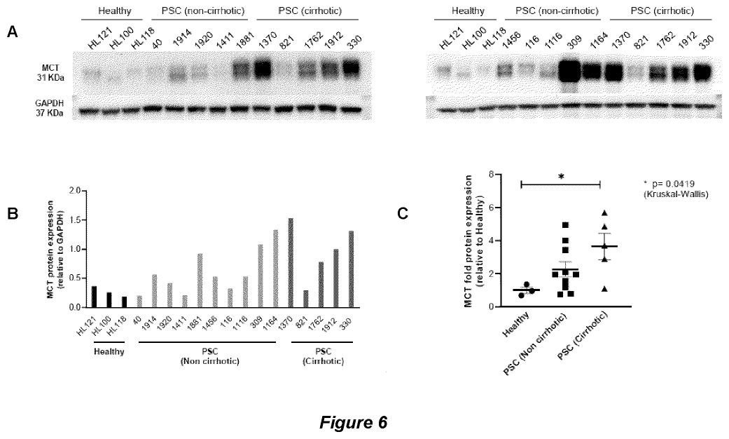

FIG. 6 shows the expression levels of MCT as reported by western blot. MCT levels were lowest in healthy tissue, increased in non-cirrhotic PSC, and highest in cirrhotic PSC (FIG. 6B). The difference between healthy and cirrhotic PSC tissues was statistically significant (FIG. 6C).

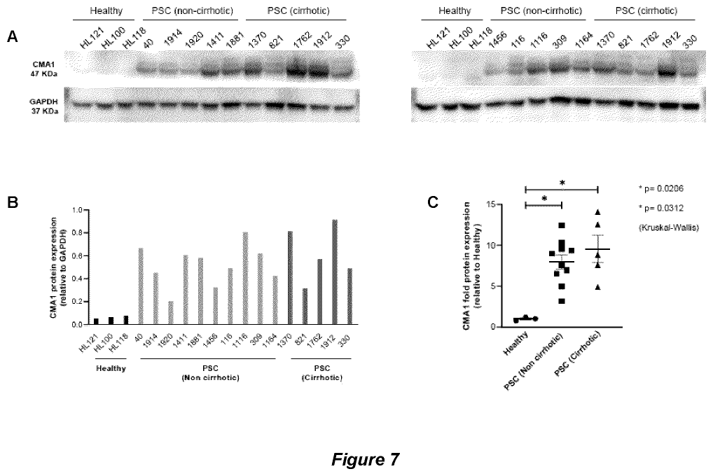

FIG. 7 shows CMA1 expression levels. Similarly, CMA1 levels were lowest in healthy tissue, increased in non-cirrhotic PSC, and highest in cirrhotic PSC (FIG. 7B). The differences between healthy and cirrhotic PSC tissues, and between healthy and non-cirrhotic PSC tissue, were statistically significant (FIG. 7C).

In summary, both CMA1 and MCT levels are increased in PSC affected tissues relative to healthy tissues, and CMA1/MCT levels may be indicators of disease progression from healthy to pre-cirrhotic to cirrhosis.

### Example 5—Chymase and Tryptase In Vitro

ECM scaffolds will be obtained from healthy and cirrhotic PSC livers.

HSC cells will be cultured on 3D ECM scaffolds. Degranulated mast cells will be added to the HSCs on the ECM, allowing the released tryptase and chymase to activate HSCs. In a parallel experiment, either a chymase inhibitor (Group 3), or a tryptase inhibitor (Group 4), or both (Group 5), will be added to the degranulated mast cells prior to adding to the HSCs. Additional control experiments will include empty vehicle (Group 1) and a positive control (Group 2). HSCs will then be analysed for markers of activation, including αSMA, Col1 and TGF-β.

Groups 3 and 4 will report lower levels of the markers of fibrosis than Group 1. Group 5 will report lower levels of the markers of fibrosis than groups 3 and 4 combined. Therefore, dual inhibition of both tryptase and chymase will reduce fibrosis to a greater extent than inhibition of each alone.

### Example 6—Therapeutic Efficacy of Chymase and Tryptase in Mouse Models of PSC In Vivo

Bile duct ligation (BDL) is a reliable and well established model of cholestatic liver diseases in rodents (Heinrich et al, 2011). C57BL/6 mice of 7-8 weeks of age will undergo surgical bile duct ligation (BDL) to induce liver cholestasis and fibrosis.

Mice will be dosed daily po (small molecule) or twice weekly iv (antibody) for 2 weeks according to Table 2. Dosing will begin 1 day after BDL.

TABLE 2

| | | | Dosing | Dosing |
|---|---|---|---|---|
| Group | # Mice | Treatment | Frequency | Duration |
| 1 | 10 | Sham control | Daily | 2 weeks |
| 2 | 15 | BDL + Vehicle | Daily | 2 weeks |
| 3 | 15 | BDL + Positive | Daily | 2 weeks |
| | | control | | |
| 4 | 15 | BDL + Chymase | BID | 2 weeks |
| | | inhibitor | | |
| 5 | 15 | BDL + Tryptase | Twice weekly | 2 weeks |
| | | inhibitor | | |
| 6 | 15 | BDL + Chymase | BID + twice | 2 weeks |
| | | inhibitor + Tryptase | weekly | |
| | | inhibitor | | |

Homozygous Mdr2 KO mice spontaneously develop severe biliary fibrosis via dysregulation of pro- and antifibrogenic genes, and are an established in vivo model of primary sclerosing cholangitis (Popov et al, 2005).

Mice will be dosed daily po (small molecule) or twice weekly iv (antibody) for 2 weeks according to Table 3. Dosing will begin at 10 or 12 weeks of age within ±3 days from their recorded date of birth.

Experimental cohorts will be as shown below:

TABLE 3

| | # | | Age at | Dosing | Dosing |
|---|---|---|---|---|---|
| Group | Mice | Treatment | Dosing Start | Frequency | Duration |
| 1 | 10 | Vehicle | 10 weeks | Daily | 2 weeks |
| 2 | 10 | Positive control | 10 weeks | Daily | 2 weeks |
| 3 | 10 | Chymase inhibitor | 10 weeks | BID | 2 weeks |
| 4 | 10 | Tryptase inhibitor | 10 weeks | Twice | 2 weeks |
| | | | | weekly | |
| 5 | 10 | Chymase | 10 weeks | BID + | 2 weeks |
| | | inhibitor + | | twice | |
| | | Tryptase | | weekly | |
| | | inhibitor | | | |
| 6 | 10 | Vehicle | 14 weeks | Daily | 2 weeks |
| 7 | 10 | Positive control | 14 weeks | Daily | 2 weeks |
| 8 | 10 | Chymase inhibitor | 14 weeks | BID | 2 weeks |
| 9 | 10 | Tryptase inhibitor | 14 weeks | Twice | 2 weeks |
| | | | | weekly | |
| 10 | 10 | Chymase | 14 weeks | BID + | 2 weeks |
| | | inhibitor + | | twice | |
| | | Tryptase | | weekly | |
| | | inhibitor | | | |

At the end of the dosing period, mice will be euthanised and the following samples collected:
a. Blood will be collected and processed for serum
b. Liver will be excised, and weights recorded

Serum samples will be analysed for the levels of liver disease biomarkers alanine transaminase (ALT), aspartate transaminase (AST), alkaline phosphatase (ALP), and gamma-glutamyltransferase (GGT) levels.

Liver samples will be analysed for fibrosis through detection of αSMA, Col1 and TGF-β.

Groups treated with a chymase inhibitor or tryptase inhibitor individually will have lower serum levels of one or more of ALT, AST, ALP and GGT levels, and reduced liver fibrosis, than observed in the corresponding vehicle treated groups. Groups treated with both a chymase inhibitor and a tryptase inhibitor will show serum levels of the biomarkers and levels of liver fibrosis which are lower than those reported for the corresponding vehicle treated groups, and lower than those for corresponding groups treated with chymase or tryptase inhibitors individually.

### Example 7—Effects of Chymase and Tryptase on Collagen Production in HSC Model

Primary human hepatic stellate cells (HSCs) were grown to confluence and maintained in growth media for 10 days. Cells were maintained in serum-free media for 48 h and subsequently stimulated with either human chymase alone (30, 100 or 300 ng/mL), human tryptase alone (300, 1000, 3000 ng/mL) or human chymase and tryptase in combination as indicated for a further 48 h. The PAR2 agonist at 10 or 100 ng/mL and TGFb1 (3 ng/mL) served as a positive control, whilst serum-free conditions provided a negative control.

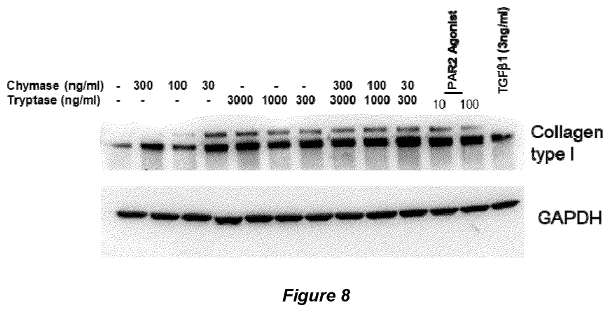

Media was removed and the cells and extracellular proteins deposited lysed in RIPA buffer. Protein extracts from the cells and extracellular proteins were resolved by western blot and probed for the ECM protein collagen type I (which is comprised of collagen 1alpha1 and 1alpha2) chains—hence the double bars seen in FIG. 8—and the loading control protein GAPDH.

The results of this experiment can be seen in FIG. 8. As can be seen, exogenous addition of chymase to HSCs induced a marked increase in collagen type I protein (lanes 2 to 4) compared to basal levels of collagen type I seen in untreated cells (lane 1). Similarly, exogenous addition of tryptase to HSCs induced a significant increase in collagen type I protein (lanes 5 to 7) compared to basal levels of collagen type I. Exogenous addition of both chymase and tryptase to HSCs induced a significant increase in collagen type I protein (lanes 5 to 7) compared to basal levels of collagen (lane 1). Thus, chymase and tryptase alone or in combination led to an increase in collagen type I levels at all concentrations assessed.

### Example 8—Chymase and Tryptase Play Separate but Complementary Roles in Collagen Deposition

Primary human hepatic stellate cells (HSCs) were grown for 2 days. Cells were maintained in serum-free media for 48 h and subsequently stimulated with either human chymase (C) at 100 ng/mL, human tryptase (T) alone at 1000 ng/mL) or in combination (C 100 ng T 1000 ng) as indicated for a further 48 h. TGFβ1 served as a positive control, and serum-free conditions (SFM) as a negative control.

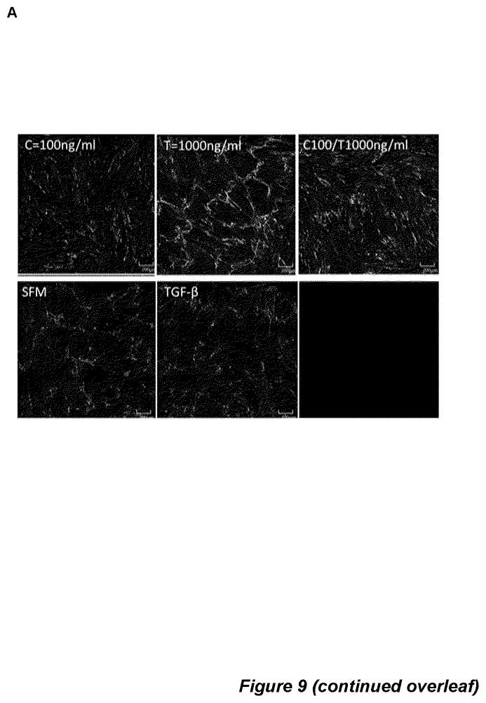

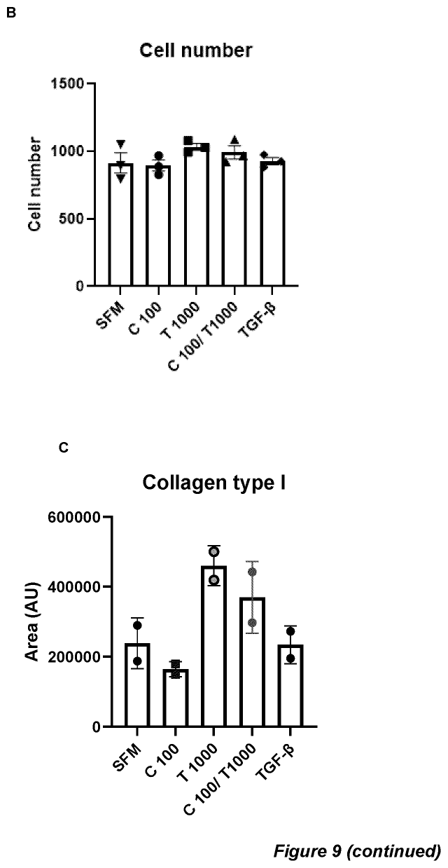

Following stimulation, media was removed and the cells washed in PBS and fixed with Formalin 4% for 15 min. Cells and extracellular proteins were stained with Rb ANTI-Col I (Novus Biologicals, NB600-408, 1:500) and images were acquired at 20× using a confocal microscope. The results are shown in FIG. 9.

None of the treatment groups resulted in a significant difference in cell number (see FIG. 9B)

Chymase (C) induced a change in distribution of deposited collagen type I compared to serum-free alone (SFM) as can be seen in FIG. 9A (top row), but did not induce a significant change in collagen area relative to SFM negative control (FIG. 9C 2^{nd} bar). In contrast, tryptase (T) induced an increase (˜2-fold) in deposited collagen type I compared to serum-free alone (SFM) at 100 ng/mL (see FIG. 9A, top row and 9C 3^{rd} bar). Interestingly, the addition of both chymase and tryptase (C100/T1000) led to an increase in collagen type I and a change in distribution (see FIG. 9A top right panel) compared to SFM (bottom left panel), alongside a marked increase in collagen type I (Figure C, 4^{th} bar). It is thus shown that chymase and tryptase have separate but complementary roles in controlling the pattern of collagen deposition. Inhibition of these separate yet complementary roles will result in significant effects on stalling, reversing or curing fibrosis.

### Example 9—Chymase and Tryptase Regulate the Secretion of Pro-Fibrogenic Factors

The effect of chymase and tryptase on the secretion of profibrogenic factors was investigated.

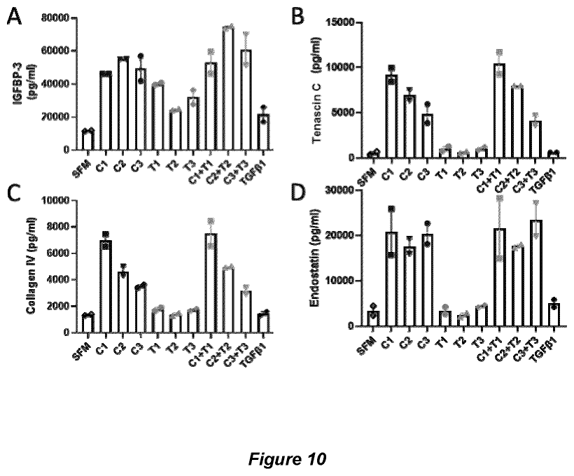

Primary human HSCs were grown to confluence and maintained in growth media for 10 days. Cells were maintained in serum-free media for 48 h and subsequently stimulated with either human Chymase (30, 100 or 300 ng/mL), human tryptase alone (300, 1000, 3000 ng/mL) or in combination with chymase for a further 48 h. The TGFβ1 (3 ng/mL) served as a positive control and serum-free conditions as a negative control. Media was removed after 48 h and levels of secreted IGFBP-3, Tenascin C, Collagen IV and Endostatin levels were determined by Luminex. Results are shown in FIG. 10.

Treatment with chymase and tryptase, alone or in combination, led to a marked increase in IGFBP3 (FIG. 10A), a known mediator of fibrosis (Flynn RS Inflamm Bowel Dis 2011), regardless of concentration.

Furthermore, exogenous addition of chymase to HSCs induced a concentration-dependent increase in the ECM proteins Tenascin C (Fig. B) and collagen IV (FIG. 10C). Chymase at all concentrations induced a significant increase in the anti-angiogenic collagen type endostatin (FIG. 10D) which is derived from the enzymatic activity of chymase on collagen. It inhibits endothelial cell proliferation and migration and stimulates endothelial cell death by apoptosis. Thus, chymase and tryptase both induce the secretion of pro-fibrotic factors, and their roles in this regulation are distinct.

### Example 10—Chymase and Tryptase Act in Concert to Increase Expression of Active TGFβ

Primary human HSCs were grown to confluence and maintained in growth media for 10 days. Cells were maintained in serum-free media for 48 h and subsequently stimulated with either human Chymase (30, 100 or 300 ng/mL), human tryptase (300, 1000, 3000 ng/mL) or both in combination as indicated for a further 48 h. The TGFb1 (3 ng/mL) served at positive control and serum-free conditions as a negative control.

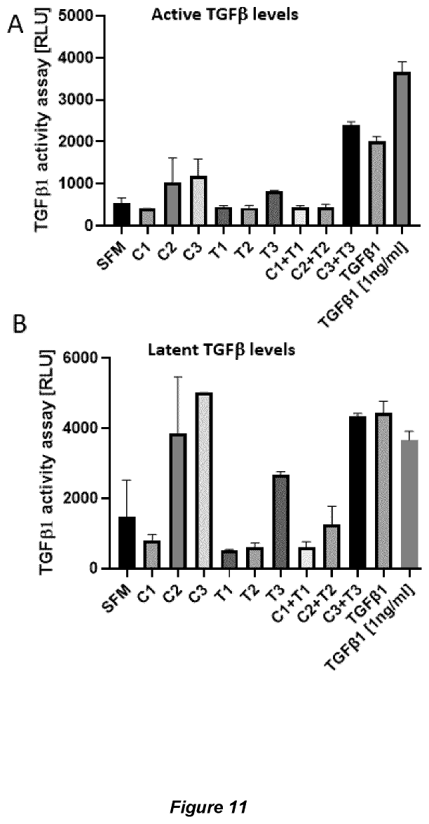

Conditioned media was collected and either transferred directly to cells harbouring a TGFb-responsive promoter linked to the firefly luciferase reporter gene for 24 h to determine the levels of active TGFb present in media, or conditioned media was heated at 80° C. for 10 minutes in order to activate in the conditioned media the latent TGFb into active TGFb present. The addition of exogenous TGFb1 (1 ng/mL served as a positive control. Cells were lysed after 24 h and levels of latent or active TGFb determined by measuring luciferase activity. The results are shown in FIG. 11.

Treatment with the combination of chymase and tryptase (C3+T3) led to a marked increase in secreted (bioavailable) active TGFβ in the conditioned media of HSCs compared to chymase (C3) or tryptase (T3) alone (see FIG. 11A). This is surprising, as in a pro-fibrotic model even additive effects above and beyond those of the TGFβ1 control would be unexpected.

In contrast, increases in secreted latent TGFβ (FIG. 11B) in the conditioned media of HSCs were seen following treatment with chymase at 100 (C2) and 30 ng/mL (C3). Whilst effects were observed for tryptase treated cells, the combination treatment was not significantly upregulated compared to cells treated with equivalent concentrations of chymase alone.

### Discussion

The involvement of mast cells in PSC is known, and has been evaluated in a range of animal models including mdr2 knock-out, bile duct ligation (BDL), methionine- and choline deficiency (MCD) and carbon tetrachloride (CCl₄) models. Mast cells promote fibrosis and vascular cell activation in bile duct ligation models (Hargrove et al, 2017), whilst suppression of mast cells through anti-histamines and cromolyn attenuates biliary proliferation and fibrosis in animal models (Jones et al, 2016).

Chymase and tryptase are secreted by mast cells, and have roles in triggering tissue remodeling and pro-fibrogenic mediators. Chymase in the ECM activates TGF-β, cytokines, MMP-9, angiotensin II and SCF (which in turn promotes mast cell secretion, resulting in a feedforward loop). Tryptase, on the other hand, activates PAR-2, chemokines, and MMPs and promotes vascular permeability. Together, activation of these factors results in inflammation, vasoconstriction and fibrosis.

Whilst the involvement of chymase has been tested in several models with chymase-specific inhibitors (Miyaoka et al, 2017), results on fibrosis readouts are modest. Similarly, tryptase inhibitors have been shown to attenuate liver fibrosis in rat bile duct ligation models (Lu et al, 2014), however this effect is insufficient to reverse fibrosis or restore diseased tissue to healthy levels. Considered alone, neither chymase nor tryptase inhibitors provide an effective treatment for stalling, reversing, or curing fibrosis.

The present application provides the first indication that chymase and tryptase are present simultaneously and act in concert to induce fibrosis, and that whilst they act in concert to regulate collagen type I protein production they do so through different mechanisms. By inhibiting chymase and tryptase in combination, the progression of fibrosis can be stopped and even reversed.

### REFERENCES

A number of publications are cited above in order to describe and disclose the invention more fully and the state of the art to which the invention pertains. Full citations for these references are provided below. The entirety of each of these references is incorporated herein.
Lazaridis N K and LaRusso N F, Primary Sclerosing Cholangitis; New England Journal of Medicine (2016); 375:1161-1170
Mendes F D, Jorgensen R, Keach J, et al. Elevated serum IgG4 concentration in patients with primary sclerosing cholangitis. Am J Gastroenterol 2006; 101:2070-2075
Bedossa P, Poynard T. An algorithm for the grading of activity in chronic hepatitis C. The METAVIR Cooperative Study Group. Hepatology. 1996; 24:289-293
Gallwitz M, Hellman L. Rapid lineage-specific diversification of the mast cell chymase locus during mammalian evolution. Immunogenetics. 2006; 58:641-654.
Doggrell S A, Wanstall J C. Vascular chymase: pathophysiological role and therapeutic potential of inhibition. Cardiovasc Res. 2004; 61:653-662.
Doggrell S A, Wanstall J C. Cardiac chymase: pathophysiological role and therapeutic potential of chymase inhibitors. Can J Physiol Pharmacol. 2005; 83:123-130.
Takai S, Jin D, Ohzu M, Tanaka K, Miyazaki M. Chymase inhibition provides pancreatic islet protection in hamsters with streptozotocin-induced diabetes. J Pharmacol Sci. 2009; 110:459-465.
Heuston S, Hyland N P. Chymase inhibition as a pharmacological target: a role in inflammatory and functional gastrointestinal disorders? Br J Pharmacol. 2012; 167(4):732-740.
Xie Q, Zhou X, Huang P, Wei J, Wang W, Zheng S. The performance of enhanced liver fibrosis (ELF) test for the staging of liver fibrosis: a meta-analysis. PLoS One. 2014; 9(4):e92772. Published 2014 Apr. 15.
Popov, Yury et al. Mdr2 (Abcb4)−/− mice spontaneously develop severe biliary fibrosis via massive dysregulation of pro- and antifibrogenic genes. Journal of Hepatology, Volume 43, Issue 6,1045-1054
Lu J, Chen B, Li S, Sun Q. Tryptase inhibitor APC 366 prevents hepatic fibrosis by inhibiting collagen synthesis induced by tryptase/protease-activated receptor 2 interactions in hepatic stellate cells. International Immunopharmacology, Volume 20, Issue 2, June 2014, Pages 352-35
Miyaoka, Yuta & Jin, Denan & Tashiro, Keitaro & Komeda, Koji & Masubuchi, Shinsuke & Hirokawa, Fumitoshi & Hayashi, Michihiro & Takai, Shinji & Uchiyama, Kazuhisa. (2017). Chymase inhibitor prevents the development and progression of non-alcoholic steatohepatitis in rats fed a high-fat and high-cholesterol diet. Journal of Pharmacological Sciences. 134.
Jones, H., Hargrove, L., Kennedy, L., Meng, F., Graf-Eaton, A., Owens, J., Alpini, G., Johnson, C., Bernuzzi, F., Demieville, J., DeMorrow, S., Invernizzi, P., & Francis, H. (2016). Inhibition of mast cell-secreted histamine decreases biliary proliferation and fibrosis in primary sclerosing cholangitis Mdr2(−/−) mice. Hepatology (Baltimore, Md.), 64(4), 1202-1216.
Hargrove L, Kennedy L, Demieville J, et al. Bile duct ligation-induced biliary hyperplasia, hepatic injury, and fibrosis are reduced in mast cell-deficient Kit^{W-sh} mice. Hepatology. 2017; 65(6):1991-2004.
Frances & Meininger (2010), A review of mast cells and liver disease: What have we learned? Dig Liver Dis. 2010 August; 42(8):529-36.
Jarido, V., Kennedy, L., Hargrove, L., Demieville, J., Thomson, J., Stephenson, K., & Francis, H. (2017). The emerging role of mast cells in liver disease. American journal of physiology. Gastrointestinal and liver physiology, 313(2), G89—G101.
Heinrich S, Georgiev P, Weber A, Vergopoulos A, Graf Rand Clavien PA. Partial bile duct ligation in mice: a novel model of acute cholestasis. Surgery 2011; 149: 445-451.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A method of treating or preventing a disease or condition associated with or characterised by fibrosis, comprising administering an effective amount of a chymase inhibitor simultaneously or sequentially with an effective amount of a tryptase inhibitor to a subject in need thereof.

2. A chymase inhibitor for use in a method of treating or preventing a disease or condition associated with or characterised by fibrosis, wherein the chymase inhibitor is administered in an effective amount to a subject in need thereof simultaneously or sequentially with an effective amount of a tryptase inhibitor.

3. A tryptase inhibitor for use in a method of treating or preventing a disease or condition associated with or characterised by fibrosis, wherein the tryptase inhibitor is administered in an effective amount to a subject in need thereof simultaneously or sequentially with an effective amount of a chymase inhibitor.

4. Use of a chymase inhibitor in the manufacture of a medicament for treating or preventing a disease or condition associated with or characterised by fibrosis, wherein the medicament delivers an effective amount of the chymase inhibitor, and the medicament is administered to a subject in need thereof simultaneously or sequentially with an effective amount of a tryptase inhibitor.

5. Use of a tryptase inhibitor in the manufacture of a medicament for treating or preventing a disease or condition associated with or characterised by fibrosis, wherein the medicament delivers an effective amount of the tryptase inhibitor, and the medicament is administered to a subject in need thereof simultaneously or sequentially with an effective amount of a chymase inhibitor.

6. The method, chymase inhibitor for use, tryptase inhibitor for use, or use according to any previous claim, wherein the disease or condition is characterised by increased expression of chymase and/or tryptase in a tissue or organ affected by fibrosis.

7. The method, chymase inhibitor for use, tryptase inhibitor for use, or use according to any previous claim, wherein the disease or condition is characterised by mast cell accumulation in a tissue or organ affected by fibrosis.

8. The method, chymase inhibitor for use, tryptase inhibitor for use, or use according to any one of claims 1 to 7, wherein the disease or condition is associated with or characterised by fibrosis of the liver, the heart, the kidney, the pancreas, the skin, the intestine, and/or the lung.

9. The method, chymase inhibitor for use, tryptase inhibitor for use, or use according to any previous claim, wherein the disease or condition is a liver disease or condition associated with or characterised by fibrosis of the liver.

10. The method, chymase inhibitor for use, tryptase inhibitor for use, or use according to claim 9, wherein the liver disease or condition is associated with or characterised by cholestasis.

11. The method, chymase inhibitor for use, tryptase inhibitor for use, or use according to claim 9, wherein the liver disease or condition is associated with or characterised by cirrhosis.

12. The method, chymase inhibitor for use, tryptase inhibitor for use, or use according to claim 9, wherein the liver disease or condition is selected from: primary sclerosing cholangitis (PSC), alcoholic liver disease (ALD), primary biliary cirrhosis (PBC), non-alcoholic steatohepatitis (NASH), and biliary atresia.

13. The method, chymase inhibitor for use, tryptase inhibitor for use, or use according to claim 9, wherein the liver disease or condition is primary sclerosing cholangitis (PSC).

14. The method, chymase inhibitor for use, tryptase inhibitor for use, or use according to claim 13, wherein PSC is associated with autoimmune hepatitis.

15. The method, chymase inhibitor for use, tryptase inhibitor for use, or use according to claim 13, wherein PSC is associated with elevated IgG4 level.

16. The method, chymase inhibitor for use, tryptase inhibitor for use, or use according to claim 9, wherein the liver disease or condition is associated with or characterised by portal hypertension.

17. The method, chymase inhibitor for use, tryptase inhibitor for use, or use according to any previous claim, wherein the tryptase inhibitor is an inhibitor of Tryptase alpha-1 and/or Tryptase beta-1.

18. The method, chymase inhibitor for use, tryptase inhibitor for use, or use according to any previous claim, wherein the tryptase inhibitor and/or the chymase inhibitor is an antibody or a fragment thereof.

19. The method, chymase inhibitor for use, tryptase inhibitor for use, or use according to any previous claim, wherein the tryptase inhibitor is operably linked to the chymase inhibitor.
